(19) United States
(12) Patent Application Publication
(10) Pub. No.: US 2026/0268483 A1
(43) Pub. Date: Sep. 10, 2026

(54) SYSTEM AND METHOD FOR MONITORING PERFORMANCE OF A MEDICAL IMAGE PROCESSING MODEL

(71) Applicant: GE Precision Healthcare LLC, Waukesha, WI (US)

(72) Inventors: Ponnam Mahendhar Goud, Bengaluru (IN); Gurunath Reddy Madhumani, Bengaluru (IN); Chitresh Bhushan, Schenectady, NY (US); Dattesh Shanbhag, Bengaluru (IN)

(73) Assignee: GE Precision Healthcare LLC, Waukesha, WI (US)

(21) Appl. No.: 19/075,046

(22) Filed: Mar. 10, 2025

(51) Int. Cl. G06T 7/00; G16H 30/40

(52) U.S. Cl. CPC G06T 7/0012; G16H 30/40; G06T 2207/20081

(57) ABSTRACT

A method and system for monitoring a medical image processing model is provided, wherein the medical image processing model is configured to process a medical image of a patient to perform at least one predefined medical image processing task. At least one trained model is configured to identify occurrence of a predefined plurality of failures for the at least one predefined medical image processing task based on outputs of the medical image processing model and to generate a text description of each identified failure. An output of the medical image processing model is provided as an input to the trained model, which generates a text description of at least one failure identified in the output. The text description of the at least one failure is stored in a model log file for the medical image processing model, wherein the model log file includes only text descriptions providing model performance information.

### BACKGROUND

The present disclosure generally relates to medical imaging, and specifically to monitoring performance of a medical image processing model.

Various models are configured for medical image processing tasks, such as to facilitate diagnosis of a patient based on medical images generated by a medical imaging modality. Further, medical image processing models are trained for facilitating various tasks during imaging, such as for assisting with pre-scanning, scan prescription, and image reconstruction tasks. Exemplary medical imaging modalities include magnetic resonance imaging (MRI), computerized tomography (CT), ultrasound, x-ray, and others. Exemplary medical image processing tasks include classification, semantic segmentation, panoptic segmentation, grounding objects, and predicting measurements. For instance, a medical image processing model may be configured to perform object identification and/or image segmentation on an MRI image of a patient's spine (or portion thereof) to identify portions of an image representing each of several vertebrae. As another example, a medical image processing model may be configured to perform image segmentation and/or edge detection on an MRI image of a patient's brain to identify one or more features or lobes of the brain. As yet further examples, a medical image processing model may be configured to perform lesion detection on PET images of a patient anatomy or to compute the volume of a liver and/or segment the lobes of a liver on CT image(s).

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

In one aspect of the disclosure, a method of monitoring a medical image processing model is provided, wherein the medical image processing model is configured to process a medical image of a patient to perform at least one predefined medical image processing task. The method includes providing at least one trained model, wherein the at least one trained model is configured to identify the occurrence of a predefined plurality of failures for the at least one predefined medical image processing task based on outputs of the medical image processing model and to generate a text description of each identified failure. The method further includes receiving an output of the medical image processing model generated in response to input of the medical image of the patient and providing each output of the medical image processing model as an input to the at least one trained model. The at least one trained model generates text description of at least one failure identified in the output, and the text description of the at least one failure is stored in a model log file for the medical image processing model. The model log file for the medical image processing model is then outputted.

In one embodiment, the method further includes generating a training dataset comprising a set of text descriptions of a plurality of failures and a set of medical images that coincide with the set of text descriptions, and training at least one of a vision-language model and a large language model with the training dataset to generate the at least one trained model.

In one embodiment, the at least one trained model includes a trained vision-language model configured to receive the output of the medical image processing model and to identify the occurrence of the predefined plurality of failures.

In another embodiment, the at least one trained model includes a trained language model configured to generate the text description of each identified failure, wherein the trained language model is trained based on a set of text descriptions and visual embeddings generated by a trained vision-language model, wherein the set of text descriptions includes at least one text description for each of the plurality of failures.

In another embodiment, the at least one trained model includes a trained image encoder of the trained vision-language model and a trained language model configured to generate the text description of each identified failure, and wherein generating the text description of the at least one failure includes providing the output of output of the medical image processing model to the trained image encoder and providing the visual embedding generated by the trained image encoder to the trained language model.

In another aspect of the disclosure, a system for monitoring a medical image processing model is provided, wherein the medical image processing model is configured to process a medical image of a patient to perform at least one predefined medical image processing task. The system includes a non-transitory memory and one or more hardware processors coupled to the non-transitory memory and configured to read instructions from the non-transitory memory to cause the system to perform at least the following operations: i) storing at least one trained model, wherein the at least one trained model is configured to identify occurrence of a predefined plurality of failures for the at least one predefined medical image processing task based on outputs of the medical image processing model and to generate a text description of each identified failure; ii) receiving an output of the medical image processing model generated in response to input of the medical image of the patient; iii) providing each output of the medical image processing model as an input to the at least one trained model; iv) generating, via the at least one trained model, a text description of at least one failure identified in the output; v) storing the text description of the at least one failure in a model log file for the medical image processing model; and vi) outputting the model log file for the medical image processing model, wherein the model log file does not include the medical image of the patient or the output of the medical image processing model.

In one embodiment, the at least one trained model includes a trained language model configured to generate the text description of each identified failure, wherein the trained language model is trained based on a set of text descriptions and visual embeddings generated by a trained vision-language model, wherein the set of text descriptions includes at least one text description for each of the plurality of failures.

In one embodiment, the at least one trained model further includes a trained image model of the trained vision-language model configured to generate the visual embeddings, and wherein generating the text description of the at least one failure includes providing the output of output of the medical image processing model to the trained image encoder model and providing the visual embedding generated by the trained image encoder model to the trained language model.

In another embodiment, the medical image of the patient is of a predetermined anatomical region of the patient obtained via a predetermined imaging modality, and wherein the predefined medical image processing task performed by the medical image processing model includes image segmentation of the medical image. Optionally, at least a subset of the plurality of failures identifies an anatomical location within the anatomical region, wherein the text description for each of the subset of the plurality of failures names the anatomical location and wherein the medical image representing each of the subset of the plurality of failures shows the respective failure in the respective anatomical location.

In another aspect of the disclosure, a method of training a vision-language model to monitor a medical image processing model is provided, wherein the medical image processing model is configured to process a medical image of a patient to perform at least one predefined medical image processing task. The method includes defining a plurality of failures for the at least one predefined medical image processing task, generating a set of text descriptions, wherein the set of text descriptions includes at least one text description for each of the plurality of failures, and generating a set of medical images that includes a medical image representing each text description in the set of text descriptions, and generating a training dataset comprising the set of text descriptions and the set of medical images correlated in image-text pairs. The method further includes training the vision-language model with the training dataset to minimize symmetric losses to decrease a distance between the correlated image-text pairs and maximize the distance between uncorrelated medical images and text descriptions, and outputting the trained vision-language model.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a diagram of an exemplary system for monitoring performance of a medical imaging model according to one embodiment of the present disclosure.

FIGS. 2A and 2B show exemplary trained models utilized in a system for monitoring performance of a medical imaging model according to embodiments of the present disclosure.

FIG. 3 illustrates an exemplary text description and a corresponding medical image comprising part of a training dataset for training at least one model according to some embodiments of the present disclosure.

FIG. 4 illustrates a performance testing of a monitoring system according to the present disclosure.

FIG. 5 illustrates another exemplary text description and a corresponding medical image comprising part of a training dataset for training at least one model according to some embodiments of the present disclosure.

FIG. 6 is a flow chart illustrating one embodiment of a method of training at least one model to monitor a medical image processing model according to one embodiment of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of “top,” “bottom,” “front,” “rear,” “left,” “right,” “horizontal,” “vertical,” and “longitudinal” features and/or relative motion, e.g., movement “up” and “down,” is generally intended as a description only of the orientation of such features relative to a reference frame of a particular example or illustration. Correspondingly, for example, a “top” feature may sometimes be disposed below a “bottom” feature (and so on), in some arrangements or embodiments. Additionally or alternatively, embodiments may be arranged in a different orientation such that “top” and “bottom” features are arranged horizontally relative to each other, for example in a “left-to-right” orientation.

The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Deep-learning-based solutions are being increasingly deployed for medical image processing and medical imaging control to accelerate pre-scanning, scan prescription, image reconstruction and image analysis. These deep learning models for medical image processing, once deployed, can experience impacts on performance due to data drift, unknown pathologies, or changes in data distribution compared to what they were trained for. For manufacturer of imaging modalities and or providers of such medical image processing models, it is important to understand how a deployed medical image processing model performs in clinical practice. Additionally, monitoring performance of medical image processing model may be required from a regulatory standpoint, which typically encourages real-world performance reporting of model effectiveness in clinical practice. Accordingly, the inventors have recognized that systems and methods are needed to automate performance monitoring of medical image processing models in real time and logging the monitoring information.

The inventors have recognized that one challenge with monitoring performance of medical image processing models is that restrictions on transmitting health information may limit the ability to log and transmit outputs of some medical image processing models and the corresponding medical images, particularly those containing images of patients or any information that could be regulated by privacy laws. Thus, there may be an inability to use model-op assessment tools that require access to ground truth information. Moreover, while bare quantitative information about model performance may be available, for example, reporting dice scores for segmentation models, such information is insufficient for fully assessing and understanding model performance.

In view of the foregoing needs and challenges in the relevant art recognized by the inventors, they developed this disclosed system and method for monitoring a medical image processing model. The disclosed system and method are configured to automatically provide textual descriptions of model performance. These textual descriptions are specific to the predefined medical image processing task that the medical image processing model is configured to perform and provide succinct and meaningful descriptions of model performance in clinical deployments. The system and method are configured to log these text descriptions into a model log file for the medical image processing model, wherein the model log file contains only text information that is divorced from any patient information or patient images and can be shared with a manufacturer's of imaging systems, other product developers, and/or regulatory bodies without any concerns regarding privacy or other regulated information. The use of text descriptions as presented herein make interpretability easy, such as for a non-technical person to ascertain model performance with respect to the predefined medical image processing task at hand.

The disclosed system and method utilize focused models to ascertain AI model performance in real-world applications. A trained model, or system of trained models, is configured to identify the occurrence of a predefined plurality of failures for at least one predefined medical image processing task based on outputs of the medical image processing model being deployed in the field, and to generate a text description of each identified failure. An output of the medical image processing model being monitored is provided as an input to at least one trained model, which may be one trained model or a system comprising multiple trained models configured to work together, that is configured to identify at least one failure of the medical image processing model and to generate a text description of the identified failure. The text description of the at least one failure, which is a plain language description of the identified failure in the image processing task, is stored in a model log file for the medical image processing model. The model log file includes only text descriptions providing model performance information and does not include the model output or any patient image data.

In one embodiment, the system is configured to image embeddings with text template descriptions via a trained multimodal vision-language model. In one embodiment, contrastive language-image pre-training (CLIP) training is used to generate rich correlated visual and text-correlated embeddings, and thus to generate the trained multimodal vision-language model. In some embodiments, the trained vision-language model may be stored and utilized for monitoring the medical images processing model. In other embodiments, the trained image encoder portion of the trained vision-language model may be used in conjunction with a specially-trained large language model (LLM) to monitor the medical image processing model, wherein the trained image encoder is configured to receive an output of the medical image processing model and to generate the visual embedding, and wherein the trained large language model is configured to receive the visual embedding generated by the trained image encoder and to generate the text description of the identified failure.

The training data for training the vision-language model (e.g., the CLIP training) and/or for training the LLM is generated by carefully understanding model success and failures in its performance of the predefined medical image processing task(s). A plurality of failures and/or a plurality of successes for a particular medical imaging model and defined and are synthesized to generate training data. The training data includes a set of text descriptions and a corresponding set of medical images. The set of text descriptions are accurate plain language descriptions of successes and/or failures of the model at the predefined medical image processing task that it was designed to perform. The set of medical images are images that represent the same successes and/or failures of the model that are described in the set of text descriptions, where the set of medical images includes an image correlating to each text description such that the training dataset comprises correlated image-text pairs. For example, the set of medical images may be synthesized to show successes and/or failures of the model and to have the same format as the output of the medical image processing model to be monitored

Notably, the disclosed system and method are distinguishable from general-purpose image captioning, which is not suitable for model monitoring since the tasks and the associated performance variables might not be well captured in general-purpose captioning. The disclosed system, being trained as described herein for detecting a predefined plurality of failures that are generated based on known failures of the model in performing the predefined medical image processing task(s), is targeted, accurate, and unlikely to hallucinate. Thereby, the system described herein is reliable for monitoring medical image processing models and will not generate errant information about model performance that could lead to detrimental changes in the medical image processing model and detrimental patient outcomes. General-purpose image captioning, on the other hand, is prone to hallucinations and is likely to miss detailed aspects that are relevant to reporting model performance.

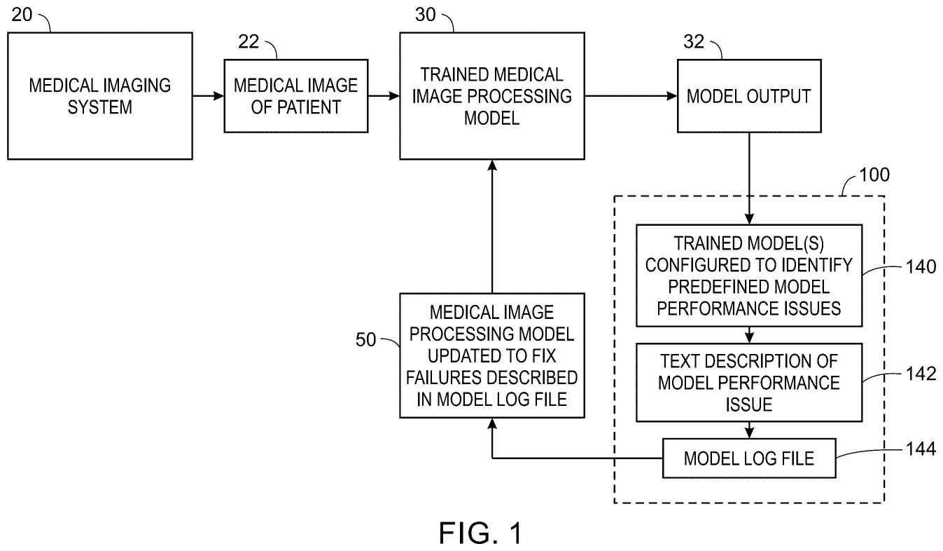

FIG. 1 is a diagram of an exemplary imaging system 20 and corresponding image processing model 30, and an exemplary system 100 for monitoring performance of the medical imaging model 30 according to one embodiment. The system 100 for monitoring performance of the medical imaging model 30 includes at least one trained model 140, such as a trained vision-language model and/or a trained LLM generated by the methods described herein. The trained model(s) 140 are configured to output a text description 142 of model performance, which is a plain language description of one or more predefined failures and/or successes that have been defined for the medical image processing task performed by the trained medical image processing model 30 being deployed in clinical practice and being monitored by the system 100.

The imaging system 20 may be, for example, an MRI, an x-ray imager, a CT imager, an ultrasound imager, or any other imaging system using an imaging modality to generate medical images 22 of a patient for medical assessment and/or diagnosis purposes. Each medical image 22 is processed by a trained medical image processing model 30 configured to perform at least one predefined medical image processing task. For example, the medical image processing model 30 may be configured to perform object recognition, image segmentation, edge detection, or other image processing tasks that are useful in identifying and/or assessing patient physiologies captured in the images, providing qualitative or quantitative image quality assessments, or other medical image processing tasks.

The model output 32 is formatted according to the predefined medical image processing task performed by the image processing model 30. For example, the model output 32 may be a processed image (such as containing a mask, bounding box, and/or edge demarcation) or other output, such as object localization, tracking, and/or size estimation (e.g., lesion localization and tracking, or other region or object estimation and tracking.

The system 100 is specifically configured and trained to receive the model output 32, whatever its format, and to identify the predefined plurality of failures, which are also defined based on the medical image processing task performed by the image processing model 30. Namely, the at least one model 140 is trained to identify the occurrence of the predefined plurality of failures and to generate a text description of each identified failure. The text descriptions 142 outputted by the at least one model 140 are stored in a model log file 144, which is a file storing the outputs of the model(s) 140 generated as it monitors performance of the medical image processing model 30. Thereby, a product developer or engineering team tasked with maintaining the medical image processing model 30 being deployed in clinical practice can review performance of that model 30, receive any performance failures or other issues described in the model log file 144, and improve/revise the model 30 as needed to more accurately process the medical images 22 of patients. Accordingly, an updated medical image processing model 50 may be generated to fix performance issues identified in the model log file 144. Since the model log file 144 does not contain the model output 32 or any patient image data, a full assessment and correction of a performance issue identified in the model log file 144 may require access to the model output 32 or similar outputs generated by simulation, which may be done on site or by other secure means that comply with privacy regulations. The updated medical image processing model 50 may replace the original trained medical image processing model 30 in the field. The updated model may then be monitored by the system 100 to continue to maintain and improve performance of the predefined medical image processing task by the trained medical image processing model 30/50.

Accordingly, the operation of the medical image processing model 30 is monitored by the system 100 over time as it operates in the field to process medical images of multiple patients, and the monitoring data (the model log file 144) generated by the system 100 can be regularly outputted and transmitted for various purposes without concern of medical data privacy regulations. In addition to maintenance and updates to the model 30, the text descriptions 142 of the model performance issues can be utilized for reporting to regulatory bodies, for research purposes, etc., without concern of privacy since no image data or other information taken from a patient or identifiable to a patient is captured in the model log file 144.

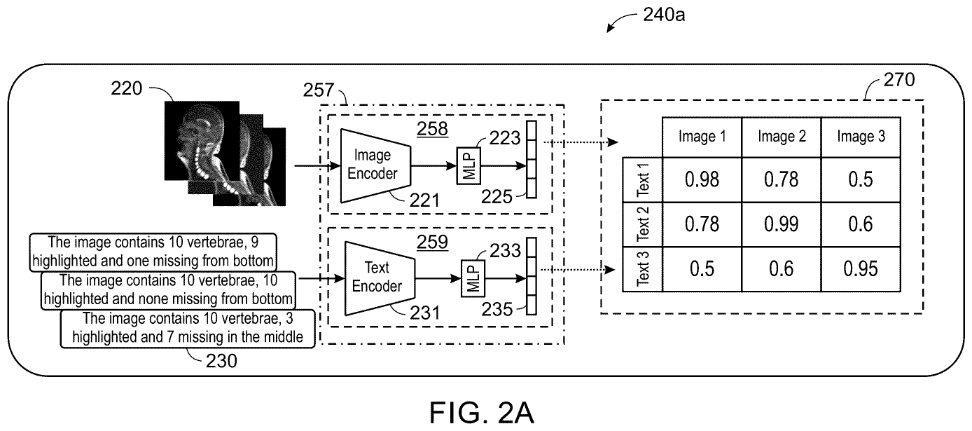

FIG. 2A is a diagram of an exemplary trained model 240a for monitoring performance of a medical imaging model, where here the trained model 240a is a vision-language model 257. FIG. 2B illustrates a second embodiment wherein a set of trained models 240b for monitoring performance of a medical imaging model is provided. The set of trained models 240b includes a large language model 260 trained on rich correlated visual and text embeddings that are generated by the trained vision-language model 257. The set of trained models 240b deployed for monitoring performance of a medical imaging model includes the trained image encoder module 258 portion of the trained vision-language model 257 and the trained LLM 260, which are used together to identify the predefined model performance issues and generate the text descriptions 242.

In FIG. 2A, the vision-language model 257 is trained on a training dataset generated specifically for the medical image processing model being monitored and the at least one predefined medical image processing task being performed. The disclosed training method is designed to generate a system 100 (FIG. 1) that will accurately and reliably assess the medical image processing model 30 in the field and will not hallucinate. The vision-language model 257, such as a CLIP model generated by CLIP training, is trained to identify that predefined plurality of performance issues in the output of the medical image processing model and to generate text descriptions of the performance issues that occur as the medical image processing model is operating in the field.

The training dataset comprises a set of text descriptions 230 that describe performance issues of the predefined medical image processing task(s) performed by the image processing model to be monitored. The set of text descriptions describe each of a plurality of failures that may occur with the model, which are known potential problems that may appear in the output of the model to be monitored. The set of text descriptions 230 is thus specific to the task performed by the model (e.g., image segmentation, object identification, etc.) and also specific to the anatomy being imaged (e.g., spine, brain, abdomen, chest/heart, shoulder or other joint, hand or other limb, or other anatomical region of the patient) and the imaging modality of medical images being processed by the model to be monitored (e.g., MRI, CT, x-ray, ultrasound, etc.). Thus, the text descriptions 230 are specific to the predetermined anatomical region of the patient and the predetermined imaging modality that the monitored medical image processing model is trained to process, as well as the specific image processing task(s) performed by the medical image processing model. For example, the text descriptions may describe specific expected failures for spine image segmentation and/or spine object identification, or brain image segmentation and/or brain object identification, depending on the task(s) being performed by the medical image processing model. The text descriptions in the set of text descriptions 230 describe all of the expected failures and/or successes that will be detected by the vision-language model 257, including describing each failure in the task(s) being performed by the medical image processing model and each anatomical location where that failure occurs. Alternatively or additionally, the set of text descriptions 230 may include a text description for each success in the task(s) being performed by the medical image processing model and/or each anatomical location where that success occurs. In the example in FIG. 2A, the text descriptions 230 are merely exemplary, which are for image segmentation to identify cervical spine vertebrae in MRI images of the cervical spine anatomical region of patients.

A set of medical images 220 is generated that includes a medical image that represents and coincides with each text description in the set of text descriptions 230. The set of medical images 220 shows the range of performance issues of the predefined medical image processing task(s) that are described in the set of text descriptions. Thus, the set of medical images 220 show each of the plurality of failures that may occur with the model, and are specific to the predetermined anatomical region of the patient (e.g., spine, brain, abdomen, chest/heart, shoulder or other joint, hand or other limb, or other anatomical region of the patient) and the predetermined imaging modality (e.g., MRI, CT, x-ray, ultrasound, etc.) that the monitored medical image processing model is trained to process, as well as the specific image processing task(s) (e.g., image segmentation, object identification, etc.) performed by the medical image processing model.

The vision-language model 257 comprises a image encoder module 258 and a text encoder module 259. The image encoder module 258 comprises an image encoder 221 and a neural network 223 (referred to here as the “first” neural network). The image encoder 221 is configured to encode the set of medical images 220 into a plurality of encoded image samples, which are provided to the first neural network 223. The first neural network 223 is trained to output image embeddings 225 based on the encoded image samples generated by the image encoder 221. The first neural network 223 may be, for example, a multilayer perceptron (MLP). The text encoder module 259 is structured similarly, comprising a text encoder 231 and a neural network 233 (referred to here as the “second” neural network). The text encoder 231 is configured to encode the set of text descriptions 230 into a plurality of encoded text samples, which are provided as inputs to the second neural network 233. The second neural network 233 is trained to receive the output of the text encoder 231 and output text embeddings 235.

The vision-language model 257 is trained with the training dataset to minimize symmetric losses to decrease a distance between the correlated image-text pairs and maximize the distance between uncorrelated medical images and text descriptions—such as using contrastive language-image pre-training (“CLIP” training). The set of text descriptions 230 and the set of medical images 220 are arranged in image-text pairs to form the training dataset. The image-text pairs are provided as parallel image and text inputs to the image encoder module 258 and the text encoder module 259, respectively. Thus, the image encoder module 258 is provided with and trained on each image in the set of medical images 220 while the text encoder module 259 is provided with and trained on each text description in the set of text descriptions 230, where the inputs are provided as parallel image-text pairs. The outputs of the image encoder module 258 and the text encoder module 259 are compared via an image-text contrastive loss function 270 which is configured to assess the cosine similarity between the outputs of the modules 258 and 259 and the image text pairs in the training data and to provide feedback accordingly to minimize symmetric losses—i.e., to decrease a distance between the correlated image-text pairs and maximize the distance between uncorrelated medical images and text descriptions.

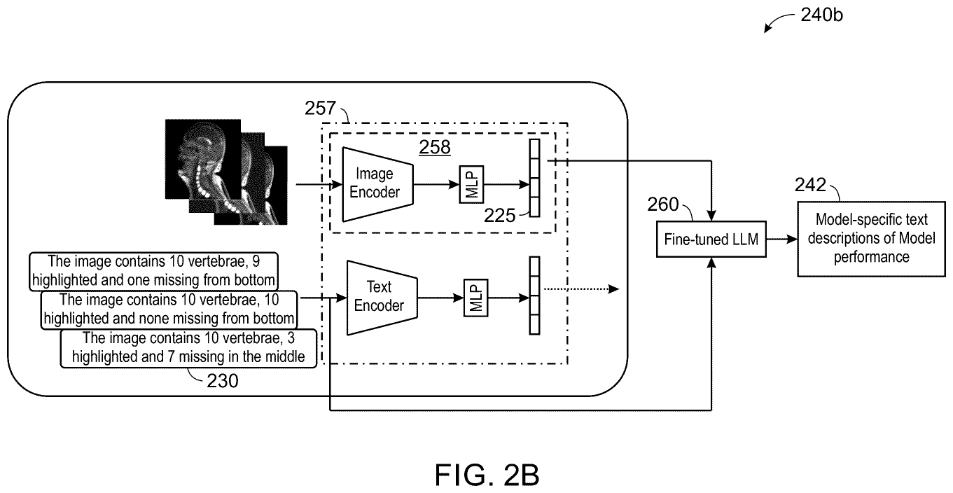

The training dataset comprises the set of text descriptions 230 and the set of medical images 220 arranged in image-text pairs. The image-text pairs represent the full range of performance issues that the vision-language model 257 will be trained to detect and describe. FIG. 3 illustrates an exemplary image-text pair 320a and 330a for training a vision-language model to monitor performance of a spine image segmentation model. Here, the medical image processing model to be monitored is configured to perform image segmentation to label pixels of an MRI image of a cervical spine associated with spinal vertebrae. The output of the spine image segmentation model to be monitored is spine MRI images containing masks labeling multiple vertebrae. This spine segmentation model may be configured to process all spine MRI images (e.g., cervical, thoracic, lumbar, and/or full spine images) or may be configured to process just cervical MRI images. The vision-language model 257 is trained accordingly so that it can process all outputs and monitor performance in all image domains where the spine segmentation model is employed. The text description 330a describes an exemplary failure of the spine segmentation model, which is that “1 vertebra is missing in the middle.” It also describes an exemplary success, which is that it successfully highlighted 10 vertebrae in the cervical station image. The image 320a corresponds with the text description 330a, showing a cervical station image with a mask 325 labeling ten out of eleven vertebrae with one mask missing over a vertebra location 326 in the middle. The image may be a real output of the spine segmentation model or may be a simulated output that has been generated to match the text description and show the failure/success. Image-text pairs of this sort, showing each failure and each success to be identified, would be generated to create the training dataset for monitoring this spine segmentation model. In various implementations, the training dataset may comprise hundreds, thousands, or even tens of thousands or more of image-text pairs.

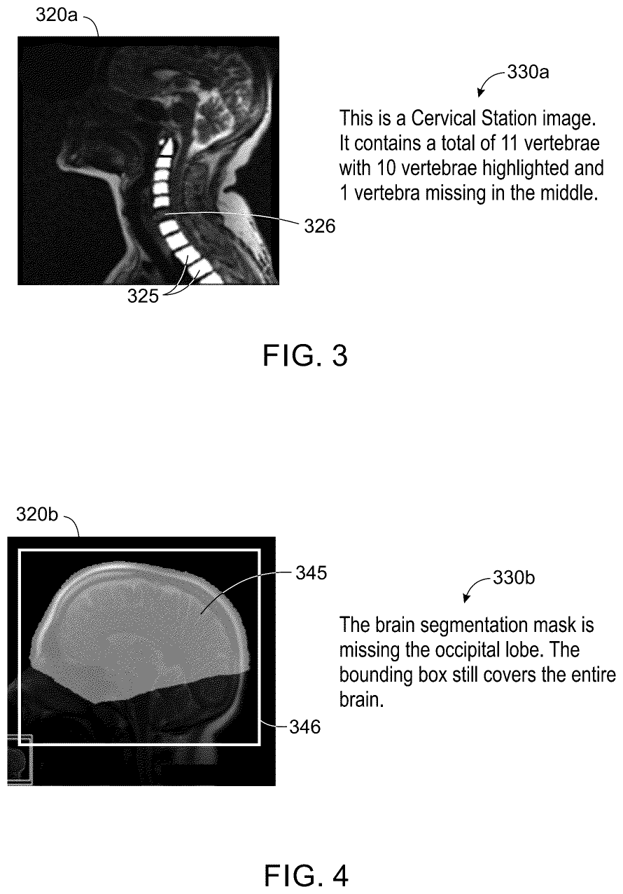

FIG. 4 illustrates another exemplary image-text pair 320b and 330b for training a vision-language model to monitor performance of a brain object recognition and segmentation model. In this example, the medical image processing model to be monitored is configured to perform object recognition of the brain in an MRI image of a brain associated, generating a bounding box 346 that encompasses all identified pixels associated with the brain object. The medical image processing model to be monitored is also configured to perform image segmentation to label pixels associated with lobes of the brain. The output of the brain image segmentation model to be monitored is brain MRI images containing bounding boxes around the brain and a mask labeling pixels associated with the lobes of the brain. The vision-language model 257 is trained accordingly so that it can process all outputs and monitor performance of both the object recognition task and the image segmentation task. The text description 330a describes the failure that “the brain segmentation mask is missing the occipital lobe.” It also describes an exemplary success, which is that it successfully performed the object recognition in that “the bounding box covers the entire brain.” The image may be a real output of the brain object recognition and segmentation model, or may be a simulated output that has been generated to match the text description and show the failure/success. Image-text pairs of this sort, showing each failure and each success to be identified, would be generated to create the training dataset for monitoring this brain object recognition and segmentation model. In various implementations, the training dataset may comprise hundreds, thousands, or even tens of thousands or more of such image-text pairs.

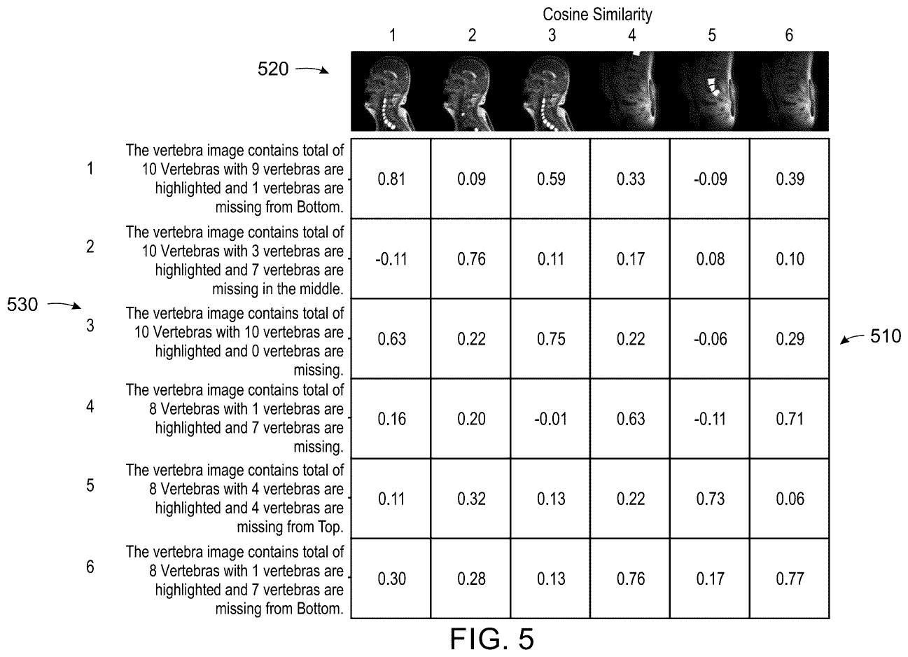

FIG. 5 illustrates exemplary output correlations from a trained vision-language model trained to process spine segmentation model trained according to the methods described herein. The cosine similarity measure schedule 510 shows the correlation outputs from the trained vision-language model between each of the text descriptions in the set of text descriptions 530 (which, for purposes of visual illustration, is shown here as containing only six descriptions but would in practice contain many hundreds or thousands of text descriptions) and each of several images outputted by the spine segmentation model. The figure shows good performance in accurately correlating text descriptions of failures in spine image segmentation with corresponding labeled MRI spine images presenting those failures. The cosine schedule 510 between the medical image outputs of the spine segmentation model and the text templates shows that, in most of the cases, the images are well correlated with the text (wherein the corresponding diagonal contains the highest values). The depicted cosine schedule 510 was generated based on randomly selected output images from the spine image segmentation model and text from the training dataset and performed correlation between them. Notice that in text #4, there is a higher correlation to last image (0.71), while there is similar similarity scores for images #4 and #6. This makes sense since text #6 adds only a “bottom” keyword to text #4. This suggests that there is good overlap between each text description and the corresponding image (e.g., station, total and missing highlighted vertebrae, and location).

Returning to FIG. 2B, another embodiment of the at least one model for monitoring a medical image processing model is provided. Here, the trained vision-language model 257 is used to train a large language model (LLM) to output the text descriptions of the model performance issues. First, the trained vision-language model 257, such as a CLIP model, is generated using the above-described training data and methods to identify the predefined plurality of performance issues. Thereafter, an LLM 260 is trained to correlate the text embedding outputs of the image encoder module 258 with the text descriptions in the predefined set of text descriptions 230 and to generate model-specific text description of model performance on the plurality of predefined successes and failures captured in the training data. Thus, the LLM 260 is trained on rich correlated visual and text embeddings generated by the vision-language model 257. This constrained training will produce an LLM 260 that will accurately and reliably generate text descriptions and will not hallucinate. The trained image encoder module 258 and the trained LLM 260 are then used together as a set of models 240b configured to monitor the clinical operation of the medical image processing model.

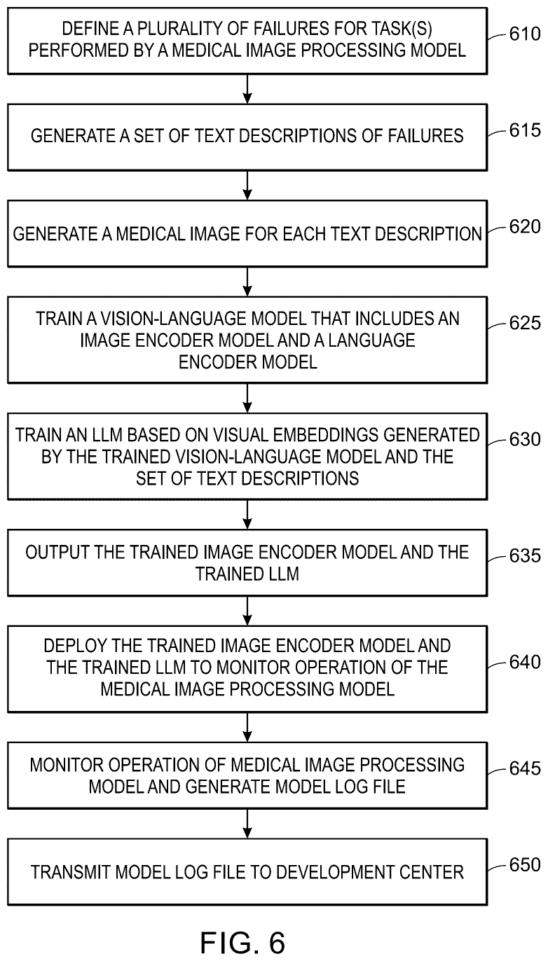

FIG. 6 is a flow chart illustrating an exemplary method of training at least one model to monitor a medical image processing model. First, a plurality of failures are defined at step 610 for tasks performed by the medical image processing model. In some embodiments, a plurality of successes may also be defined. A set of text descriptions are generated at step 615, which includes at least one text description for each failure in the plurality of failures and each success in the plurality of successes. In some implementations, at least a subset of the plurality of failures identifies an anatomical location within the anatomical region, and the text description for each of the subset of the plurality of failures names the anatomical location and wherein the medical image representing each of the subset of the plurality of failures shows the respective failure in the respective anatomical location. A medical image for each text description is generated at step 620. The medical image and corresponding text description are then correlated into image-text pairs to generate the training data.

A vision-language model is then trained on the training data at step 625, wherein the vision-language model includes an image encoder model and a text encoder model (e.g., the trained image encoder module 258 and the trained text encoder module 259 described with respect to FIGS. 2A and 2B). Training the vision-language model may include encoding, by an image encoder model, the set of medical images generated at step 620 into a plurality of encoded image samples and, by a text encoder model, the set of text descriptions into a plurality of encoded text samples. The plurality of encoded image samples are then processed with first neural network (e.g., a first MLP) to generate visual encodings and processing the plurality of encoded text samples with second neural network (e.g., a second MLP) to generate text encodings, wherein training the vision-language model includes comparing the visual encodings and the text encodings and computing the symmetric losses corresponding to the image-text pairs using a contrastive loss function.

A language model is then trained at step 630 based on visual embeddings generated by the trained vision-language model and the set of text descriptions in the set of text descriptions generated at step 615. The trained image encoder model and the trained language model are then outputted at step 635 as a set of models that get deployed (represented at step 640) to generate a model log file tracking the performance of the medical image processing model. The trained image encoder model and the trained language model are then utilized at step 645 to process and monitor the outputs of the medical image processing model, wherein the text descriptions outputted by the trained language model are stored in a model log file for the monitored model. The model log file is then transmitted, at step 650, to the entity and/or location tasked with maintaining the medical image processing model so that the logged text descriptions can be utilized to assess whether there are performance issues with the model. As described above, the model log file does not contain patient images or patient data, and thus transmitting the model log file does not risk transgressing regulations regarding patient information or raise privacy concerns.

It should be noted that, as used herein, the term module or mechanism can encompass hardware, software, firmware, or any suitable combination thereof. In various embodiments, any suitable computer-readable media can be used for storing instructions for performing functions and/or processes described herein. For example, in some embodiments, computer-readable media can be transitory or non-transitory. For example, non-transitory computer-readable media can include media such as magnetic media (such as hard disks, floppy disks, etc.), optical media (such as compact discs, digital video discs, Blu-ray discs, etc.), semiconductor media (such as RAM, Flash memory, electrically programmable read-only memory (EPROM), electrically erasable programmable read-only memory (EEPROM), etc.), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

This written description uses examples to disclose the invention(s), including the best mode, and also to enable any person skilled in the art to make and use the invention(s). Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention(s) is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A method of monitoring a medical image processing model, wherein the medical image processing model is configured to process a medical image of a patient to perform at least one predefined medical image processing task, the method comprising:
providing at least one trained model, wherein the trained model is configured to identify occurrence of a predefined plurality of failures for the at least one predefined medical image processing task based on outputs of the medical image processing model and to generate a text description of each identified failure;
receiving an output of the medical image processing model generated in response to input of the medical image of the patient;
providing each output of the medical image processing model as an input to the at least one trained model;
generating, via the at least one trained model, a text description of at least one failure identified in the output;
storing the text description of the at least one failure in a model log file for the medical image processing model; and
outputting the model log file for the medical image processing model.

2. The method of claim 1, wherein the model log file does not include the medical image of the patient or the output of the medical image processing model.

3. The method of claim 1, wherein the at least one trained model includes a trained vision-language model configured to receive the output of the medical image processing model and to identify the occurrence of the predefined plurality of failures.

4. The method of claim 1, wherein the at least one trained model includes a trained language model configured to generate the text description of each identified failure, wherein the trained language model is trained based on a set of text descriptions and visual embeddings generated by a trained vision-language model, wherein the set of text descriptions includes at least one text description for each of the plurality of failures.

5. The method of claim 4, wherein the at least one trained model further includes a trained image encoder model of the trained vision-language model, and wherein generating the text description of the at least one failure includes providing the output of output of the medical image processing model to the trained image encoder model and providing the visual embedding generated by the trained image encoder model to the trained language model.

6. The method of claim 1, wherein the medical image of the patient is of a predetermined anatomical region of the patient obtained via a predetermined imaging modality, and wherein the predefined medical image processing task performed by the medical image processing model includes image segmentation of the medical image.

7. The method of claim 6, wherein at least a subset of the plurality of failures identifies an anatomical location within the anatomical region, wherein the text description for each of the subset of the plurality of failures names the anatomical location and wherein the medical image representing each of the subset of the plurality of failures shows the respective failure in the respective anatomical location.

8. A system for monitoring a medical image processing model, wherein the medical image processing model is configured to process a medical image of a patient to perform at least one predefined medical image processing task, the system comprising:
a non-transitory memory; and
one or more hardware processors coupled to the non-transitory memory and configured to read instructions from the non-transitory memory to cause the system to perform operations comprising:
storing at least one trained model, wherein the at least one trained model is configured to identify occurrence of a predefined plurality of failures for the at least one predefined medical image processing task based on outputs of the medical image processing model and to generate a text description of each identified failure;
receiving an output of the medical image processing model generated in response to input of the medical image of the patient;
providing each output of the medical image processing model as an input to the at least one trained model;
generating, via the at least one trained model, a text description of at least one failure identified in the output;
storing the text description of the at least one failure in a model log file for the medical image processing model; and
outputting the model log file for the medical image processing model.

9. The system of claim 8, wherein the model log file does not include the medical image of the patient or the output of the medical image processing model.

10. The system of claim 8, wherein the at least one trained model includes a trained language model configured to generate the text description of each identified failure, wherein the trained language model is trained based on a set of text descriptions and visual embeddings generated by a trained vision-language model, wherein the set of text descriptions includes at least one text description for each of the plurality of failures.

11. The system of claim 10, wherein the at least one trained model further includes a trained image encoder model of the trained vision-language model configured to generate the visual embeddings, and wherein generating the text description of the at least one failure includes providing the output of output of the medical image processing model to the trained image encoder model and providing the visual embedding generated by the trained image encoder model to the trained language model.

12. The system of claim 8, wherein the medical image of the patient is of a predetermined anatomical region of the patient obtained via a predetermined imaging modality, and wherein the predefined medical image processing task performed by the medical image processing model includes image segmentation of the medical image.

13. The system of claim 12, wherein at least a subset of the plurality of failures identifies an anatomical location within the anatomical region, wherein the text description for each of the subset of the plurality of failures names the anatomical location and wherein the medical image representing each of the subset of the plurality of failures shows the respective failure in the respective anatomical location.

14. A method of training at least one model to monitor a medical image processing model, wherein the medical image processing model is configured to process a medical image of a patient to perform at least one predefined medical image processing task, the method comprising:
defining a plurality of failures for the at least one predefined medical image processing task;
generating a set of text descriptions, wherein the set of text descriptions includes at least one text description for each of the plurality of failures;
generating a set of medical images that includes a medical image representing each text description in the set of text descriptions;
generating a training dataset comprising the set of text descriptions and the set of medical images correlated in image-text pairs;
training the at least one model with the training dataset to identify occurrence of the plurality of failures in the set of medical images and to generate text descriptions of the plurality of failures based on the set of text descriptions; and
outputting the at least one trained model, wherein the at least one trained model is configured to process outputs of the medical image processing model to identify occurrence of any failure of the plurality of failures and to generate a text description of the identified failure.

15. The method of claim 14, wherein the at least one model includes a vision-language model, wherein the step of training the at least one model includes training the vision-language model with the training dataset to minimize symmetric losses to decrease a distance between the correlated image-text pairs and maximize the distance between uncorrelated medical images and text descriptions.

16. The method of claim 15, wherein the at least one model further includes a large language model, wherein the step of training the at least one model further includes training the large language model based on the set of text descriptions and visual embeddings generated by the trained vision-language model.

17. The method of claim 16, wherein the at least one trained model outputted includes the trained large language model and a trained image encoder model of the trained vision-language model, wherein the trained image encoder model is configured to receive an output of the medical image processing model and to generate the visual embedding, and wherein the trained large language model is configured to receive the visual embedding generated by the trained image encoder model and to generate the text description of the identified failure.

18. The method of claim 14, wherein the medical image of the patient is of a predetermined anatomical region of the patient obtained via a predetermined imaging modality, and wherein the predefined medical image processing task performed by the medical image processing model includes image segmentation of the medical image.

19. The method of claim 18, wherein at least a subset of the plurality of failures identifies an anatomical location within the anatomical region, wherein the text description for the each of the subset of the plurality of failures names the anatomical location and wherein the medical image representing each of the subset of the plurality of failures shows the respective failure in the respective anatomical location.

20. The method of claim 14, further comprising defining a plurality of successes for the at least one predefined medical image processing task, and wherein generating the set of text descriptions to further include at least one text description for each of the plurality of successes.
